(19) United States
(12) Patent Application Publication
(10) Pub. No.: US 2008/0213183 A1
(43) Pub. Date: Sep. 4, 2008

(54) Free or Liposomal Gemcitabine Alone or in Combination with Free or Liposomal Idarubicin

(76) Inventors: Marcel Bally, Bowen Island (CA); Nancy Dos Sanios, Richmond (CA); Euan Ramsay, Vancouver (CA)

Correspondence Address: MORRISON & FOERSTER LLP, 12531 HIGH BLUFF DRIVE, SUITE 100, SAN DIEGO, CA, 92130-2040, US

(21) Appl. No.: 11/575,655

(22) PCT Filed: Sep. 20, 2005
(86) PCT No.: PCT/CA05/01434

§ 371 Date: May 15, 2008

Related U.S. Application Data
(60) Provisional application No. 60/610,969, filed on Sep. 20, 2004.

(51) Int. Cl. A61K 49/00; A61K 9/127

(52) U.S. Cl. 424/9.2; 424/450

(57) ABSTRACT

The use of the maximum tolerated dose (MTD) of individual drugs to determine appropriate administration ratios of drugs for combination therapy, wherein the ratios of drugs are fixed based on the same percentage of the MTD for each drug. Furthermore, antineoplastic compositions comprising liposomal encapsulated gemcitabine alone or in combination with free or liposomal encapsulated antineoplastic agents, such as idarubicin, irinotecan, etopside, cisplatin, cyclophosphamide, doxorubicin, or vincristine are diclosed.

### CROSS-REFERENCE TO RELATED APPLICATIONS

This application claims benefit of provisional application U.S. Ser. No. 60/610,969 filed 20 Sep. 2004. The contents of this application are incorporated herein by reference.

### TECHNICAL FIELD

The invention relates to determination of ratios of drugs that when used in combination treatment will be non-antagonistic. More particularly, the invention is directed to providing a ratio that is reflected in the maximum tolerated dose of each drug, and in particular in the formulation it is administered. In another aspect, the invention relates to the development of liposomally encapsulated gemcitabine alone or in combination with other drugs useful for disease therapy.

### BACKGROUND ART

The administration of combinations of drugs to treat various conditions has a long history, in particular in the treatment of cancer. One difficulty in employing this approach is to ensure that the drugs are administered in a ratio that is non-antagonistic. Under these circumstances, the dosage level of each drug may be lowered from that otherwise required, and, especially in the instance where the drugs in the combination operate by independent mechanisms, the overall effectiveness of treatment is greatly enhanced.

PCT publication WO 03/028696 describes one approach to assure that non-antagonistic ratios of combinations of drugs are maintained at the site of their action. This is achieved by administering the drugs associated with delivery vehicles such that the pharmacokinetics are controlled by these vehicles, not by the drugs themselves. The appropriate ratio of the active agents in the vehicles is verified by in vitro assessment of biological effect in appropriately selected cell lines and providing ratios that remain non-antagonistic over a wide range of concentrations. One algorithm employed to determine the appropriate ratio is the Chou-Talalay approach as described, for example, in Chou, T. C., et al., Ed. Adv. Enzyme Regul. (1984) 22:27.

The present invention offers an alternative approach to determining the appropriate ratio for administration of combination drugs. In the case of the present invention, the drugs may be administered as free agents or may be associated with particulate delivery vehicles, such as liposomes.

Although applicants are unaware of compositions wherein gemcitabine is entirely encapsulated in liposomes, a previous study by Moog, R., et al., Cancer Chemother. Pharmacol. (2002) 49:356 considered compositions wherein 33% of the gemcitabine was encapsulated in vesicular phospholipid gels whereas 67% of the gemcitabine was in free form. This composition showed a dose reduction of 40-60 fold as compared to free drug.

### DISCLOSURE OF THE INVENTION

This invention describes a method of treating disease with a combination of two or more drugs at a fixed dose. The method of treating disease may prevent, delay progression or cure cancer, either the primary tumor or metastatic lesions which have disseminated to other locations in the body. Alternatively, the disease may be rheumatoid arthritis or other autoimmune disorders including transplant organ rejection.

The choice of drug combinations employs prior knowledge of any overlap in drug mechanism, drug targeting and drug toxicity and ADME characteristics. Thus, the drugs to be combined in treatment are generally those whose activities are expected to complement each other. According to the invention, the selected drugs are provided in a ratio that is determined by fixing the ratio at a particular level of the maximum tolerated dose for each drug in the formulation in which it is to be supplied. Selection of a fixed dose combination enables one to ‘fix’ the optimal effect of the drug combination. In one preferred embodiment, both drugs are then co-formulated in a manner such that the two drugs can be administered in a single procedure or composition.

In one aspect, therefore, the invention is directed to a method to determine a desirable ratio of two or more drugs to be administered in the treatment of a disease or other undesired condition, which method comprises preparing a composition, or designing a protocol in which each drug is present at the same percentage of its maximum tolerated dose in the subject to be treated. Each drug may be supplied at 100%, 90%, 80%, 66%, 60% or 50% of its maximum tolerated dose (MTD) or at any fixed percentage that is identical for all drugs in the combination including the specific values set forth above, and lower values, e.g. 30% as well. In another aspect, a desired ratio of one or more drugs in combination for preparation of a composition or for design of a protocol is determined by use of an animal model wherein the ratio of amounts of drugs to be administered in the animal model is determined as described in the previous aspect, and verified to be antagonistic in the animal model. Adjustments may be made to the ratio, then, to improve the effects as shown in the animal model to determine the final design of the composition or protocol.

The foregoing two methods of determining appropriate drug ratios result in appropriate compositions for administration and appropriate protocols. Thus, other aspects of the invention relate to the compositions so designed and to methods of treating diseases or conditions using the compositions and protocols so designed.

In still another aspect, the invention relates to liposomal formulations of gemcitabine, as applicants believe that gemcitabine has not heretofore been formulated in this manner. As demonstrated herein, formulation of gemcitabine in liposomes results in a significant increase in its effectiveness. The invention thus also relates to combinations of liposomal gemcitabine with other drugs, such as idarubicin and other anthracyclines, cisplatin and other platinum-based compounds, and various other anti-neoplastic agents.

The drugs in fixed dose compositions may consist of a free form of the drug or a pharmaceutically acceptable salt or hydrate thereof. In one embodiment, one or both compounds may be present in a liposomal formulation. The liposomal formulation can be selected by those skilled in the art of liposomally encapsulating drugs. For example a DSPC/CH/PEG (50:45:5 mole ratio) liposome formulation is one liposomal formulation for gemcitabine. In addition, the liposome may be modified to selectively target specific organs or sites of disease.

In one embodiment, one compound in a combination is gemcitabine optionally in liposomal formulation with a drug selected from for example: etoposide, cisplatin, cyclophosphamide, doxorubicin, vincristine or idarubicin. In one embodiment the combination comprises liposomal gemcitabine in combination with liposomal idarubicin. One fixed dose composition of free gemcitabine and idarubicin is 334 and 2 mg/kg, respectively. A fixed dose composition for liposomal gemcitabine and liposomal idarubicin is 3.4 and 2 mg/kg, respectively.

The fixed dose combination can be further combined with radiation or surgery to treat cancer. Additional agents may include small molecules, monoclonal antibodies and/or nucleic acid based therapies.

### BRIEF DESCRIPTION OF THE DRAWINGS

FIGS. 1A and 1B show cytotoxic activity of gemcitabine and idarubicin and combinations thereof on P388 lymphocytic leukemia cells.

FIGS. 2A and 2B show dose reduction index analysis at an IC90 of idarubicin (IDA) and gemcitabine (GEM) used alone or in combination (A) and the combination index of GEM/IDA (1:10) fixed molar ratio (B).

FIG. 3 shows plasma elimination of free and liposomal gemcitabine in Balb/c mice.

FIG. 4 shows P388 antitumor activity of a single i.v. bolus injection of free and liposomal gemcitabine administered at maximum tolerated dose (MTD).

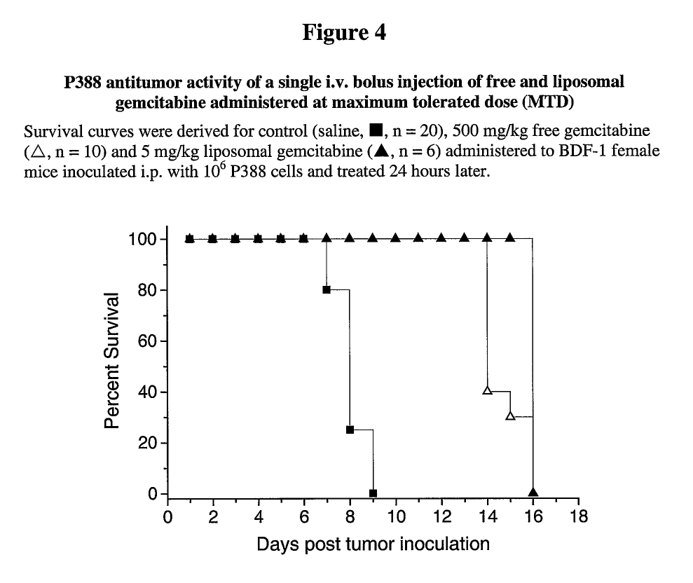

FIG. 5 shows antitumor activity of free and liposomal idarubicin and gemcitabine combination treatment.

### MODES OF CARRYING OUT THE INVENTION

In one aspect, the invention is directed to methods to determine appropriate ratios of drug combinations for treatment of conditions or diseases. In the invention method, the ratio is based on the maximum tolerated dose of each drug in the combination. As used herein, “maximum tolerated dose” (MTD) is defined in terms of the subject to be treated. When animal model determinations are employed, the dose is defined as the maximum dose that could be administered wherein no animal in the group shows signs of significant toxicity for at least 30 days after drug treatment.

In one embodiment of the invention method, the composition or protocol to be administered to a subject is designed based on a fixed percentage of the maximum tolerated dose of each drug in either an animal model or in the course of phase I studies where the subject to be treated is human. As noted above, the resulting composition or protocol employs a dosage of each drug which is the same fixed percentage of the MTD. In an alternate method, this is used as a starting point in an animal model, and the ratio is modified to optimize the results in the animal model, such as a murine, rabbit, or other model. The MTD employed in these methods is that for the formulation that will be used in the composition or protocol; thus if liposomal compositions or other particulate vehicle compositions are used in the protocol, it is the MTD for that formulation that is employed in the invention method.

As a hypothetical example, for a combination of drug A with a maximum tolerated dose of 100 mg/kg and a drug B with an MTD of 50 mg/kg, the invention method would encompass employing these drugs in a ratio of 2:1—e.g., 75 mg/kg:37.5 mg/kg or 50 mg/kg:25 mg/kg. If the MTD for drug A in liposomal formulations is reduced to 25 mg/kg, the numerical value of the ratio will be reversed at the selected levels.

With regard to the aspect of the invention which employs gemcitabine, the importance of this drug is noted as follows:

Gemcitabine is 2′2′-difluoro-deoxycytidine analogue, bearing structural similarity to cytosine arabinoside. The prodrug gemcitabine becomes activated following phosphorylation by deoxycytidine kinase. The di-phosphorylated derivative of gemcitabine, dFdCDP, has been shown to be a strong inhibitor of ribonucleotide reductase leading to a decrease of the deoxyribonucleotide pools for DNA synthesis. The tri-phosphorylated derivative, dFdCTP, is incorporated into DNA during the synthesis (S) phase of the cell cycle, inhibiting the action of DNA polymerases leading to a block in DNA synthesis. Primer extension assays indicated that one nucleotide is added subsequent to the addition of gemcitabine into a newly synthesized DNA strand, rendering gemcitabine less susceptible to removal by the exonuclease function of DNA polymerases.

Gemcitabine has antitumor activity in both haematological and solid tumor models, including leukemia, lung (non small cell), pancreatic, breast, ovarian and bladder. In comparison to cytosine arabinoside, gemcitabine is more cytotoxic, and has longer retention in tumor tissue, higher accumulation within leukemia cells, and a higher binding affinity for deoxycytidine kinase.

Gemcitabine is also relatively well-tolerated; the dose limiting toxicity is myelosuppression and this is short lived with no need for hematopoietic growth factors. Other adverse, yet transient, side effects include fever, rash and elevated liver function tests including aspartate aminotransferase and alanine aminotransferase enzymes. Gemcitabine's non-overlapping toxicities with many other drug classes make it an ideal candidate for combination therapy, often without the need for dose reduction.

Gemcitabine is currently licensed as frontline therapy for the treatment of non small cell lung and pancreatic cancers. Although gemcitabine has reasonable response rates when administered alone, higher response rates were observed when gemcitabine was combined with other classes of drugs. In non small cell lung cancer activity a dose of 800-1250 mg/m2 achieved overall response rates ranging from 20% (when used as a single agent) (Gatzemeier, U., et al., Eur. J. Cancer (1996) 32A:243, Anderson, H., et al., J. Clin. Oncol. (1994) 12:1821) to 50% when used in combination with cisplatin with median survival greater than 1 year (Abratt, R. P., et al., J. Clin. Oncol. (1997) 15:744). More recently, the combination of doxorubicin and gemcitabine for the treatment of advanced breast cancer has shown favorable complete response rates in clinical trials (Jassem, J., Semin. Oncol. (2003) 30:11).

While it has been shown that it has been advantageous to encapsulate cytosine arabinoside in liposomes (Allen, T. M., et al., Cancer Res. (1992) 52:2431), the use of liposomes for delivery of gemcitabine delivery is not believed to be known.

The liposomal composition of this drug can be optimized as illustrated in the example below. As determined therein a suitable liposomal formulation is prepared from DSPC/CH/PEG at 50:45:5 mole ratio.

The resulting liposomal formulation of gemcitabine is then employed alone or in combination with other drugs, preferably according to a ratio determined by the method set forth hereinabove.

In all cases, the compositions and protocols of the invention may be administered to subjects by a variety of routes.

Administration may be, for example, intravenous, intramuscular, intraparenteral or enteral, such as oral or rectal, and parenteral administration. Subjects are mammals or other vertebrates, including man, comprising a therapeutically effective amount of at least two pharmacologically active combination partners alone or in combination with one or more pharmaceutically acceptable carrier.

The following examples are intended to illustrate but not limit the invention. In these examples, the following materials and methods are employed:

Lipids: 1,2-distearoyl-sn-glycero-3-phosphatidylcholine (DSPC) and 1,2-distearoyl-sn-glycero-3-phosphatidyl-ethanolamine (DSPE)-conjugated poly(ethylene glycol) lipids (molecular weight 2000) were obtained from Avanti Polar Lipids, Inc. (Alabaster, Ala., USA). Cholesterol (CH) was obtained from the Sigma-Aldrich Canada Ltd. (Oakville, ON, Canada).

Chemicals: HEPES (N-[2-hydroxyethyl]piperazine-N′-[2-ethanesulfonic acid]), citric acid, sephadex G-50 (medium), 3[H]-cholesteryl hexadecyl ether (CHE), OGP (n-octyl glucopyranoside) detergent, MTT (3-4,5-dimethylthaizol-2-yl)-2,5-diphenyl tetrazolium bromide) reagent, and all other chemicals were obtained from Sigma-Aldrich Canada Ltd. (Oakville, ON, Canada). Picofluoro-15 and Picofluoro-40 scintillation fluids were obtained from Packard Bioscience (Groningen, The Netherlands). Triton X-100 detergent was purchased from BioRad (Richmond, Calif., USA

Drugs: The anthracyclines idarubicin hydrochloride (10 mg idarubicin; 100 mg lactose; MW. 533.97; Pharmacia and Upjohn, Boston, Mass., USA) and gemcitabine hydrochloride (200 mg gemcitabine, 200 mg mannitol, 12.5 mg sodium acetate; MW. 299.5; Eli-Lilly Canada, Inc. Toronto, Ontario, Canada) were manufactured by the indicated companies and obtained from British Columbia Cancer Agency (Vancouver, BC, Canada). 3[H]-gemcitabine was obtained from Moravek Biochemicals Inc. (Brea, Calif., USA).

Cell Culture: Mouse serum was obtained from Cedarlane (Hornby, Ontario, Canada). Dulbecco's modified eagle's medium (DMEM), RPMI 1640 and Hank's balanced salt solution (HBSS) were obtained from StemCell Technologies Inc. (Vancouver, BC, Canada). Fetal bovine serum (FBS) was obtained from Hyclone (Logan, Utah, USA). L-glutamine and typsin-ethylenediamminetetraacetic acid (EDTA) were purchased from Gibco BRL (Life Technologies, Burlington, ON, Canada). Microtitre (96-well) Falcon□ plates, culture flasks and blood collection tubes containing liquid EDTA were obtained from Becton-Dickinson Biosciences (Mississauga, Ontario, Canada). Microfuge tubes were obtained from VWR (West Chester, Pa., USA).

Liposome Preparation: Liposome formulations were prepared by the extrusion technique. Briefly, lipids were dissolved in chloroform and mixed together in a test tube at indicated molar ratios. 3[H]-cholesteryl hexadecyl ether (CHE) was added as a non-exchangeable, non-metabolizeable lipid marker. The chloroform was evaporated under a stream of nitrogen gas and the sample was placed under high vacuum overnight to remove residual solvent. The lipid films were rehydrated in either citrate (300 mM citric acid, pH 4.0; with pH gradient for remote loading) or HBS (HEPES buffered saline, 20 mM HEPES, 150 mM NaCl, pH 7.4; no pH gradient) by gentle mixing and heating. Cholesterol-containing formulations were subjected to five cycles of freeze (liquid nitrogen) and thaw (65° C.) prior to extrusion. The newly formed multilamellar vesicles (MLV's) were passed 10 times through an extruding apparatus (Northern Lipids Inc., Vancouver, BC, Canada) containing two stacked 100 nm Nucleopore® polycarbonate filters (Northern Lipids Inc., Vancouver, BC, Canada).

QELS liposome size analysis: The mean diameter and size distribution of each liposome preparation (prior to addition of ethanol or drugs) was analyzed by a NICOMP model 270 submicron particle sizer (Pacific Scientific, Santa Barbara, Calif., USA) operating at 632.8 nm, was typically 100±30 nm.

Drug Loading: Remote loading of anthracyclines: Following hydration of lipid films in citrate (300 mM citric acid; pH 4.0), extrusion and size determination, liposomes were passed down a sephadex G-50 column (10 cm×1.5 cm) equilibrated with HBS (HEPES buffered saline; 20 mM HEPES, 150 mM NaCl, pH 7.4) to exchange the external buffer. The eluted liposomes had a transmembrane pH gradient, pH 4.0 inside and pH 7.4 outside. Drugs were added to the liposome preparation (5 mM total lipid concentration) at a 0.2 drug-to-lipid mole ratio at varying incubation temperatures.

For drug loading rate determination of anthracyclines, 100 μl aliquots were added to mini spin columns at 1, 2, 5, 10, 15, 30, 60 and 120 minutes following remote loading. Spin columns were prepared by adding glass wool to a 1 cc syringe and sephadex G-50 beads packed by centrifugation (680 g, 1 min). Following addition of the sample to the column, the liposome fraction was collected in the void volume (centrifugation 680 g, 1 min) and both lipid and drug content were analyzed. The lipid concentration was measured by 3[H]-CHE radioactive counts and drug concentration was determined by measuring the absorbance at 480 nm (HP 8453 UV-visible spectroscopy system, Agilent Technologies Canada, Inc., Mississauga, ON, Canada) in a 1% Triton X-100 solution and compared to a standard curve. Prior to absorbance analysis, samples were heated in boiling water to the cloud point of the detergent and cooled to room temperature.

Passive loading of gemcitabine: Gemcitabine hydrochloride (200 mg) was rehydrated in HBS (HEPES buffered saline, 20 mM HEPES, 150 mM NaCl, pH 7.4) at a concentration of 50 mg/ml. A lipid film (150 μmole lipid) containing trace quantities of 3[H]-CHE radiolabel was prepared and rehydrated with 1.6 ml (214 μmole gemcitabine) solution at 40° C. for 60 min. The samples were passed through an extruding apparatus containing 2 stacked 100 nm polycarbonate filters at 65° C. The mean diameter and size distribution of each liposome preparation was determined as previously mentioned. Lipid and gemcitabine concentrations were measured to estimate the encapsulation efficiency and final drug-to-lipid mole ratio. Lipid concentrations were determined by measuring radioactivity by liquid scintillation counting and gemcitabine concentration was determined by absorbance spectrophotometry with samples diluted in 10 mM OGP (n-octyl-glucopyranoside) detergent and measured at 268 nm and compared to a standard curve.

Pharmacokinetic Analysis: Balb/c mice breeders, 20-22 g, were purchased from Charles River Laboratories (St. Constant, QC, Canada) and bred in-house. Mice were housed in micro-isolator cages and given free access to food and water. All animal studies were conducted according to procedures approved by the University of British Columbia's Animal Care Committee and in accordance with the current guidelines established by the Canadian Council of Animal Care.

The plasma elimination of idarubicin and gemcitabine containing tracer quantities of 3[H]-gemcitabine was assessed. Mice were injected with 33 μmoles/kg drug administered intravenously into the lateral tail vein of Balb/c mice. At various time points up to 4 hours post drug administration, blood was collected by tail nick (collected in microfuge tubes) or cardiac puncture (collected in liquid EDTA coated tubes), centrifuged at 1000 g to isolate the plasma fraction. The plasma was placed in a separate microfuge tube and vortexed to ensure a homogenous distribution.

The tail nick procedure for obtaining blood samples was used to minimize the number of mice sacrificed. In this way, three blood samples could be obtained from a single mouse within a 24 hour time interval. In brief, the lateral tail vein of mice was nicked with a small sharp blade. A 25 μl glass pipette, pre-washed with EDTA, was used to withdraw blood. The blood was expelled into a microfuge tube containing 200 μl of 5% (wt/vol) EDTA and thoroughly mixed. Blood/EDTA samples were centrifuged for 10 minutes at 1000 g. The supernatant was transferred to a 1.5 ml microfuge tube. 250 μl Hank's balanced salt solution (HBSS) was added to the pellet, resuspended and centrifuged for 10 minutes at 1000 g. The supernatants were mixed together. Assuming a 48% hematocrit for a 20 gram Balb/c mice, approximately 13 μl plasma was obtained from a 25 μl blood sample. From the recovered plasma samples, aliquots were used to measure drug (and or lipid) concentrations.

The plasma elimination of liposomes containing tracer quantities of 3[H]-CHE or 14[C]-CHE was assessed. When required, samples were concentrated with cross-flow cartridges (500,000 MWCO) manufactured by A/G Technology Corp. (Needham, Mass., USA) prior to i.v. administration. Mice were injected with 165 μmoles/kg drug administered intravenously into the lateral tail vein of Balb/c mice. At various time points up to 24 hours post drug administration, blood was collected by tail nick (collected in microfuge tubes) and cardiac puncture (collected in liquid EDTA coated tubes), centrifuged at 1000 g to isolate the plasma fraction. Studies assessing two radiolabels, 3[H]-CHE and 3[H]-DPPC, were completed and the results demonstrated that the recovered plasma lipid concentrations were not significantly different.

The plasma elimination of liposomal drugs containing doxorubicin, daunorubicin, idarubicin, or gemcitabine samples administered intravenously into the lateral tail vein of Balb/c female mice was assessed. Mice were injected with 33 μmole/kg drug and 165 μmole/kg lipid. For liposomal gemcitabine samples, mice were injected with 33 μmole/kg gemcitabine at an approximate 0.1 drug-to-lipid mole ratio). At various time points post drug administration, blood was collected by tail nick or cardiac puncture. Plasma lipid and 3[H]-gemcitabine were quantified by liquid scintillation counting. Anthracyclines were extracted from plasma with a partitioning assay, followed by fluorescence spectrometer detection.

The plasma elimination data was modeled using WinNonlin (version 1.5) pharmacokinetic software (Pharsight Corporation, Mountain View, Calif., USA) to calculate pharmacokinetic parameters. As the plasma elimination data was not obtained from a single mouse (blood samples from 2 mice were required to measure the drug and lipid concentrations over a 24 hour time interval) the values were reported as mean plasma area-under-the-curve AUC without standard deviations, thus statistical analysis could not be performed. The mean plasma AUC for a defined time interval was determined from the concentration-time curves and subsequent calculation by the standard trapezoidal rule.

For in vitro analysis, P388 wild type and doxorubicin resistant (ADR) cells were obtained from the National Cancer Institute tissue repository (Bethesda, Md., USA) and were propagated in vivo. In brief, one vial of frozen ascites was removed from the nitrogen tank and thawed at 37° C. and cells were injected i.p. into female BDF-1 mice (6-8 weeks old, 20-22 g, Charles River Laboratories, St. Constant, QC, Canada). Transfer mice were euthanized and a peritoneal lavage was performed. With a 1 cc syringe with 20 gauge needle, 0.5-1.0 ml of peritoneal fluid was removed and aliquotted into a 15 ml falcon tube containing 5 ml of Hank's Balanced Salt Solution (HBSS, no calcium or magnesium). 0.5 ml aliquot was transferred into another 15 ml conical sterile tube containing 5 ml HBSS. The cells were exposed to plastic culture wear (for adherence of monocytes) and Ficoll-Paque density centrifugation (red blood cell removal). For cell counting, an aliquot (0.1 ml) of P388 cell suspension is diluted 1:1 with trypan blue (2%), stain and counted using the haemocytometer, only cells with >90% cell viability were used for experimentation. For each passage, 2 female BDF-1 mice were injected with 1×106 cells in 0.5 ml (2×106 cells/ml) of P388 cell suspension intraperitoneally. This was repeated every 6-8 days to a maximum of 20 passages. Cells adequate for animal experiments were used between the 3rd-20th passage. For tissue culture experiments such as MTT cytotoxicity assays, P388 cells were obtained following peritoneal lavage and treatment to remove red blood cells and peritoneal macrophages. P388 cells were maintained in RPMI culture media containing 10% FBS and 1% L-glutamine as a cell suspension in 25 cm2 culture flasks maintained at 37° C. in humidified air with 5% CO2 and subcultured by dilution daily for no more than one week.

In order to assess cytotoxicity the MTT (3-(4,5-dimethylthiazol-2-yl)-2,5-diphenyl tetrazolium bromide) assay was utilized. Cells were counted by trypan blue staining (>90% cell viability for experiments) and seeded in 96 well microtiter plates at 1500 cells/0.1 ml diluted in medium. The wells in the perimeter of the 96 well microtiter plates contained 0.2 ml sterile water. After 24 hours at 37° C., serial dilutions of drugs (including doxorubicin, idarubicin or gemcitabine) were added to the plate (100 μl/well). Control wells consisted of media only (200 μl/well), or cells and media (no treatment). There were 6 replicates (per plate) for all control and treatment groups). Following a 72 hour incubation 37° C., MTT stock solution (5 mg/ml PBS; phosphate buffered saline, pH 7.4) was diluted 1:4 with media and 50 μl was added to each well. Plates were incubated for 4 hours in humidified air with 5% CO2 at 37° C. The P388 non-adherent cells were spun down for 10 minutes at 1800 RPM. The media was aspirated off and 0.15 ml DMSO was added per well and resuspended on a plate shaker (5-10 min). The absorbance was measured at 570 nm on a MRX microplate reader (Dynex Technologies, Inc., Chantilly, Va., USA). The cytotoxicity upon drug exposure was quantified by expressing the percent cell viability for each treatment relative to untreated control cells (% control). For multiple drug exposure studies, the drug concentration required to inhibit 50% (IC₅₀) and 90% (IC₉₀) of cell growth, was compared between single and combination drug treatments. This was further analyzed by the median effects principle by Chou and Talalay, cited above.

The method by Chou and Talalay was used to distinguish between synergy, antagonism and additive effects of combined drug treatments from in vitro MTT cytotoxicity assays. This method, now provided in a software package (Calculsyn; Biosoft, Cambridge, UK), derives a median effects equation (1) to correlate drug dose and effect.

fa/fu=(D/Dm)m (1)

A dose-effect plot is generally sigmoidal relationship and the above symbols represent the following: D, dose of drug; Dm, median effect dose; fa, fraction affected dose; fu, fraction unaffected dose and m, mathematical equation above forms a linear relationship known as the Median-Effect Plot.

log(fa/fu)=m log(D)−m log(Dm) (2)

Fixed ratio combinations of idarubicin and gemcitabine were initially selected on the basis of IC₅₀ of each drug. It was assumed that idarubicin and gemcitabine have mutually exclusive mechanisms of action and thus for two drugs D1 and D2, their “combination index” or additive effects is equal to 1.

(D)1/(ED50)1+(D)2/(ED50)2=1 (3)

Thus synergy was defined by a combination index (CI) of <1 and antagonism was defined as >1. Data were reported as mean±S.D. from three separate experiments, performed in triplicate.

For in vivo testing of antitumor activity was evaluated in P388 lymphocytic leukemia model.

Dose range finding studies of free and liposomal idarubicin and/or gemcitabine were performed in non-tumor bearing female BDF-1 mice. Mice were weighed daily and monitored for signs of stress or toxicity (e.g., lethargy, scruffy coat, ataxia). The maximum tolerable dose was defined as the dose that no animal in a given group exhibited signs of significant toxicity for 30 days post drug treatment.

Efficacy studies were conducted in female BDF-1 mice injected i.p. with 106 P388 cells. Treatments commenced 24 hours post tumor cell inoculation. Treatment groups consisted of saline (control) and 0.5, 1, 2 and 3 mg/kg doses of free or liposomal idarubicin administered as a single i.v. bolus injection and between 100 to 500 mg/kg gemcitabine and 1 to 5 mg/kg liposomal gemcitabine (selected on the basis of dose range finding studies). Fixed dose ratios for combination treatments were defined on the basis of 0.66 MTD when used as a single agent. Mice were monitored daily for signs of stress and toxicity as detailed in previous paragraph. Median survival and percent weight loss was determined for each treatment. Although death was indicated as an end point, animals that showed signs of illness due to tumor progression were terminated, and the day of death was recorded as the following day.

All data values are reported as mean±standard deviation (S.D.). A standard one-way analysis of variance (ANOVA) was used to determine statistically significant differences of the means. For multiple comparisons, post-hoc analysis using the Tukey-Kramer test. Survival curves were computed using the Kaplan-Meier method. Long-term survivors (survival time>60 days) were censored, and assigned a survival time of 61 days. Treatment groups were subsequently analyzed using SPSS statistics software (SPSS Inc., Chicago, Ill., USA) and compared using a two sample log-rank test. P<0.05 was considered significant for all statistics tests.

### EXAMPLE 1

### Characteristics of Liposomes

Diameters were measured by quasi-elastic light scattering using Nicomp submicron particle sizer model 370. Samples were diluted in sterile saline, pH 7.4. The mean liposome diameters were 91.7±23.7 nm for DSPC/DSPE-PEG2000 (95:5 mole ratio) and 99.8±29.0 nm for DSPC/CH/DSPE-PEG2000 (50:45:5 mole ratio) liposomes.

### EXAMPLE 2

### In Vitro Cytotoxicity of Gemcitabine and Idarubicin

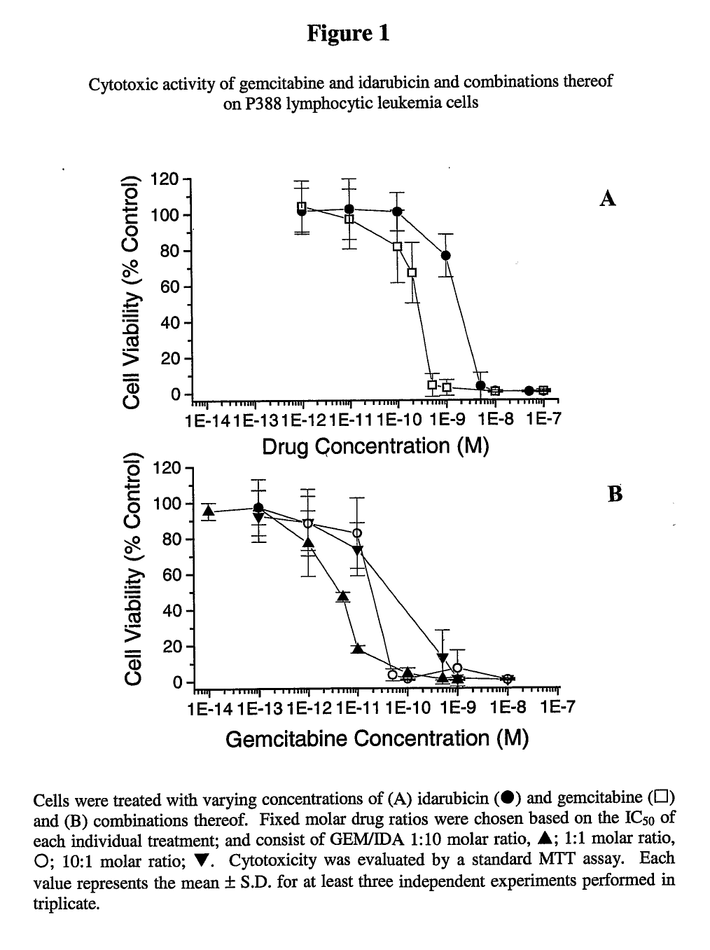

Cytotoxic activity was assessed by the standard MTT assay described above. Gemcitabine (IC₅₀=2.6×10⁻¹⁰ M) was approximately 10-fold more cytotoxic than idarubicin (IC₅₀=1.8×10⁻⁹ M) as shown in FIG. 1A. In this example, the IC₅₀ concentrations (concentration required to achieve 50% cell kill) of the individual drugs were used to define the fixed molar ratio for combination studies. Thus one molar ratio studied was set at 1:10 (GEM/IDA). In addition, 1:1 and 10:1 GEM/IDA fixed molar ratio drug combinations were also included to assess whether drug interactions were dependent on the drug molar ratio.

Cytotoxicity curves of the fixed ratio combinations of gemcitabine and idarubicin shown in FIG. 1B demonstrated a shift to the left in the cytotoxicity curves when compared to use of gemcitabine as a single agent, indicating the concentration of gemcitabine could be lowered to achieve the same effect.

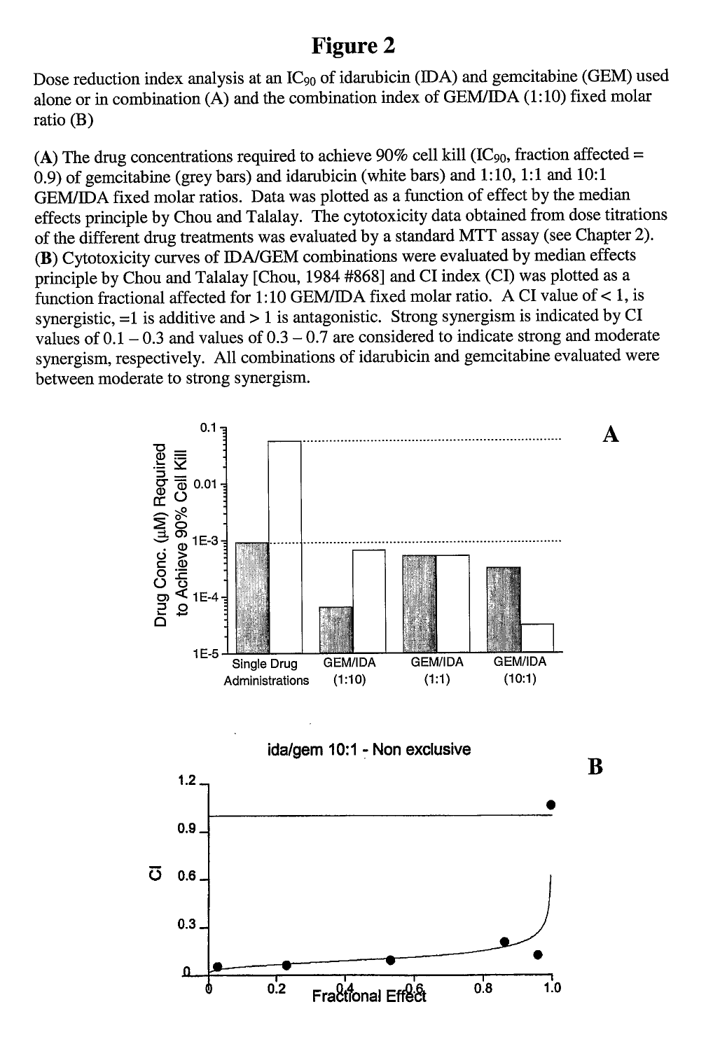

This was confirmed as shown in FIG. 2A, which summarizes the drug concentration required to achieve a 90% cell kill (fraction affected=0.9) for treatments consisting of gemcitabine or idarubicin administered alone or in combination. For treatment by either gemcitabine or idarubicin alone, the IC₉₀ drug concentrations were 0.9 nM and 5.7 nM, respectively. When P388 cells were treated with GEM/IDA at a 1:10 fixed molar ratio, less of each drug was required to achieve 90% cell kill. The fold reduction in drug concentration, also referred to as the dose reduction index (DRI), was 14 and 8.5 for gemcitabine and idarubicin, respectively. For a 1:1 GEM/IDA fixed molar ratio, the DRI was 1.8 and 11.8 for gemcitabine and idarubicin, respectively. There was a 180-fold reduction in idarubicin concentration required when administered in 10:1 GEM/IDA fixed ratio.

Dose titrations of gemcitabine and idarubicin administered alone, and in combinations added at fixed ratios were analyzed by the median effects method by Chou and Talalay to determine the combination index (CI) as a function of fraction affected (represents fraction of nonviable cells), as shown in FIG. 2B. A CI value of <1 represents synergy while a CI value of 1 or >1 indicated additive effects and antagonism, respectively. A 1:10 (GEM/IDA) fixed dose molar ratio, as well as the other ratios (data not shown), demonstrated moderate to very strong synergism, over a broad range of effective doses. This result is consistent with other reports suggesting that gemcitabine interacts synergistically with anthracyclines. Peters, G. J., et al., Pharmacol. Ther. (2000) 87:227.

### EXAMPLE 3

### Liposome Encapsulation of Gemcitabine

Previous studies indicate that liposomal idarubicin improved the median survival of mice infected with P388 leukemia cells as compared to controls and free idarubicin.

To determine if this is the case for gemcitabine, gemcitabine was passively loaded in three different liposomal formulations; DSPC/DSPE-PEG2000 (95:5 mole ratio), DSPC/CH (55:45 mole ratio) and DSPC/CH/PEG (50:45:5 mole ratio). In brief, lipid films were rehydrated with 167 mM gemcitabine (dissolved in HEPES buffered saline, pH 7.4) at 40° C. for 60 min. The samples were extruded through 2 stacked 100 nm polycarbonate filters to generate unilamellar liposomes. Two parameters were measured including liposome size by quasi-elastic light scattering (QELS) technique and encapsulation efficiency following separation of free and encapsulated gemcitabine by size exclusion chromatography. For both cholesterol-containing formulations, the mean liposome diameter ranged between 100 and 130 nm. The mean liposome diameter (57.6 mm) and encapsulation efficiency (1.8%) were significantly lower for the preparations consisting of DSPC/DSPE-PEG2000 (95:5 mole ratio). These data are shown in Table 1.

TABLE 1
Effect of lipid composition on the drug-to-lipid mole ratio and encapsulation
efficiency of passively loaded gemcitabine

| Liposome | | | | Drug-to- | |
|---|---|---|---|---|---|
| Composition | Lipid Conc. | Drug Conc. | Liposomes | Lipid | Encapsulation |
| (mole ratio) | (mM) | (mM) | size (nm) | Ratio | Efficiency (%) |
| DSPC/PEG | 100 | 167 | 57.6 (2.8) | 0.030 | 1.8 |
| (95:5) | | | | | |
| DSPC/CH | 100 | 167 | 107.0 (9.4) | 0.096 | 5.7 |
| (55:45) | | | | | |
| DSPC/CH/PEG | 100 | 167 | 101.1 (5.7) | 0.114 | 6.8 |
| (50:45:5) | | | | | |

Final drug-to-lipid mole ratios of 0.1 were obtained for the cholesterol-containing formulations, however, the DSPC/CH/PEG (50:45:5 mole ratio) liposome formulations consistently exhibited higher levels of association (˜10% improvement).

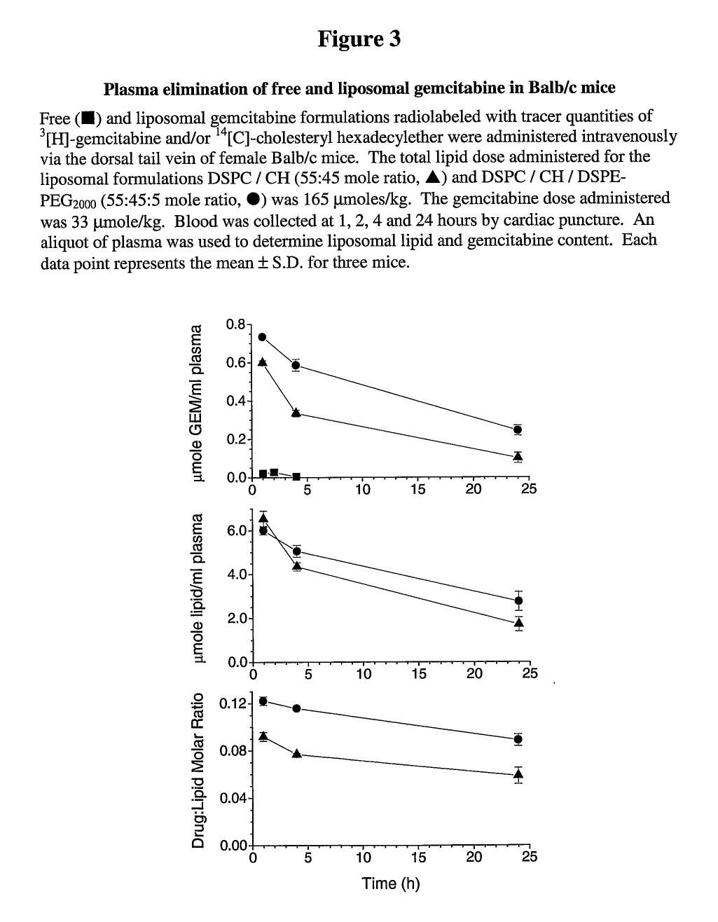

Liposome mediated increases in gemcitabine blood residence time were also evaluated as follows: Free and liposomal gemcitabine formulations were administered to female Balb/c mice at a dose of 33 μmole gemcitabine/kg (9.9 mg/kg) and 165 μmole total lipid/kg. At various time points post drug administration, blood samples were taken to measure gemcitabine and liposomal lipid plasma concentrations, and these data are shown in FIG. 3, and in Table 2.

TABLE 2
Summary of pharmacokinetic parameters of free and liposomal gemcitabine

| | AUC₀₋ₜ^{a} | T_{1/2} | Cl | AUMC | MRTₗₐₛₜ |
|---|---|---|---|---|---|
| Sample | (μmole · h · ml⁻¹) | (h) | (ml · h⁻¹) | (μmole · h² · ml⁻¹) | (h) |
| GEM | 0.1^{b} | 2.1 | 6.12 | 0.3 | 3.1 |
| DSPC/CH | 4.3^{c} | 4.4 | 0.16 | 27.1 | 6.3 |
| (50:45:5) | | | | | |
| DSPC/CH/PEG | 15.4^{c} | 14.3 | 0.05 | 319.0 | 20.7 |
| (50:45:5) | | | | | |

^{a}AUC was calculated using the trapezoidal rule (0-Tlast)
^{b}Tlast was 4 hours
^{c}Tlast was 24 hours
^{d}All pharmacokinetic elimination profiles were fit to iv-bolus one compartment model using WinNonlin Version 1.5 pharmacokinetic software. R², goodness of fit statistic for one compartment model was 0.756, 0.987 and 0.994 for free gemcitabine and liposomal gemcitabine formulations DSPC/CH and DSPC/CH/PEG, respectively.

Gemcitabine plasma concentrations were modeled using pharmacokinetic software, indicating a close fit with an i.v. bolus one compartment model.

Thus, DSPC/CH/PEG (50:45:5 mole ratio) liposomes increased plasma circulation longevity of gemcitabine more than free or liposomal DSPC/CH (55:45 mole ratio) gemcitabine. Both mean plasma area-under-the-curve (AUC) and plasma half-life (T1/2) increased 135-fold (15.4 μmole h ml-1) and 8-fold (14.3 h) when encapsulated in DSPC/CH/PEG (50:45:5 mole ratio) as compared to free gemcitabine.

### EXAMPLE 4

### Antitumor Activity of Free and Liposomal Gemcitabine in P388 Murine Leukemia

To investigate the effect of encapsulation of gemcitabine (DSPC/CH/PEG; 55:45:5 mole ratio) on therapeutic activity, efficacy experiments were performed in the P388 murine leukemia model described above. Initial dose-range finding studies performed in non-tumor bearing BDF-1 mice indicated that the maximum tolerable dose was 500 mg/kg and 5 mg/kg of free and liposomal gemcitabine, respectively. Thus, liposome encapsulation could permit a 100-fold dose reduction of gemcitabine.

At the maximum tolerable dose, 100% increase in life span (ILS) (median survival time; 16 days) was obtained in mice receiving liposomal gemcitabine, at the MTD of (5 mg/kg) and 75% ILS (median survival time; 14 days) was obtained when mice were treated with free gemcitabine at its MTD (500 mg/kg).

The maximum therapeutic dose of free gemcitabine was 400 mg/kg resulting in 87.5% ILS (median survival time; 15 days).

Thus, median survival time was enhanced for liposomal gemcitabine at a dose that was approximately 100-fold less than free drug. (This dose exhibits equivalent toxicity.)

### EXAMPLE 5

### In Vivo Determination of Drug Combination Ratios

Mice were treated with combined drugs based on a ratio defined by 66% of the individual's maximum tolerated dose (MTD). For free gemcitabine and idarubicin, 66% of MTD's are 334 mg/kg (1115 μmole/kg) and 2 mg/kg (3.8 μmole/kg), respectively. For liposomal formulations, 66% of MTD's are 3.4 mg/kg (11.4 μmole/kg) and 2 mg/kg (3.8 mg/kg) of gemcitabine and idarubicin, respectively. The results obtained when these ratios are administered in the P388 leukemia model are shown in Table 3.

TABLE 3
Antitumor activity of combinations of free and liposomal idarubicin/gemcitabine
in BDF-1 mice bearing P388 tumors

| | Drug Dose | | | | | | |
|---|---|---|---|---|---|---|---|
| | (mg/kg) | | % Weight | MST^{a} | | Cell Kill^{c} | |
| Group | IDA | GEM | Change, day 5 | (days) | % ILS^{b} | (LOG₁₀) | Survivors |
| Control | — | — | 11.8 | 8.0 | — | N/A | 0/20 |
| IDA | 0.5 | | 13.6 | 9 | 13 | 0.6 | 0/12 |
| | 1 | | 2.1 | 12 | 50 | 2.3 | 0/12 |
| | 2 | | −1.4 | 17 | 113 | 5.1 | 1/12 |
| LIDA | 0.5 | | 2.7 | 11 | 38 | 1.7 | 0/14 |
| | 1 | | 2.4 | 14.5 | 81 | 3.7 | 0/14 |
| | 2 | | −1.9 | 20.5 | 156 | ≧6 | 2/14 |
| GEM | | 100 | 0.4 | 13 | 63 | 2.9 | 0/6 |
| | | 200 | 3.0 | 14.5 | 81 | 3.7 | 0/6 |
| | | 300 | 2.3 | 14.5 | 81 | 3.7 | 0/6 |
| | | 400 | 1.8 | 15 | 88 | 4.0 | 0/6 |
| | | 500 | 0.0 | 14 | 75 | 3.4 | 0/10 |
| LGEM | | 1.0 | −4.2 | 13 | 63 | 2.9 | 0/6 |
| | | 2.5 | 3.3 | 14 | 75 | 3.4 | 0/6 |
| | | 5.0 | 1.9 | 16 | 100 | 4.6 | 0/6 |
| IDA/GEM | 0.5 | 83.5 | 0.2 | 14 | 75 | 3.4 | 0/6 |
| | 1.0 | 167 | −0.4 | 17 | 113 | 5.1 | 0/6 |
| | 2.0 | 334 | −6.2 | 18 | 125 | ≧6 | 0/6 |
| LIDA/GEM | 0.5 | 83.5 | −2.4 | 14 | 75 | 3.4 | 0/6 |
| | 1.0 | 167 | −2.8 | 16.5 | 106 | 4.9 | 0/6 |
| | 2.0 | 334 | −1.2 | 20.5 | 156 | ≧6 | 1/6 |
| IDA/LGEM | 0.5 | 0.85 | 1.8 | 14 | 75 | 3.4 | 0/6 |
| | 1.0 | 1.7 | 1.4 | 18 | 125 | 4.9 | 0/6 |
| | 2.0 | 3.4 | 0.5 | 19.5 | 144 | ≧6 | 1/6 |
| LIDA/LGEM | 0.5 | 0.85 | 1.7 | 16.5 | 106 | 4.9 | 0/6 |
| | 1.0 | 1.7 | 3.9 | 19 | 138 | ≧6 | 0/6 |
| | 2.0 | 3.4 | 1.8 | 30 | 281 | ≧6 | 1/6 |

^{a}MST, median survival time
^{b}Percent increase in lifespan (ILS) values were determined from median survival times comparing treated and saline control groups
^{c}Log cell kill, represents the number of cells killed from treatment based on median survival. The correlation between median survival and number of inoculated cells were determined in a separate study. For efficacy studies mice were inoculated with 10⁶ P388 cells, treatment commenced 24 hours following inoculation. Thus a log cell kill ~4 indicates 10² cells remaining.

An increase in median survival times was observed for mice administered the liposomal drug combination, 30 days (281% ILS), as compared to the free drug combination, 18 days (125% ILS). Drug induced weight loss was less than 5% in both of these treatments. The data shown in Table 3 indicate that free gemcitabine combined with LIDA (2 mg/kg) resulted in improved therapeutic effects, but the combined effect was only 50% of that noted when the liposomal drugs were combined. Similar conclusions can be drawn by comparing the % ILS values observed at the highest doses of free drug combinations (% ILS=125), liposomal idarubicin/free gemcitabine (% ILS=156) and free idarubicin/liposomal gemcitabine (% ILS=144).

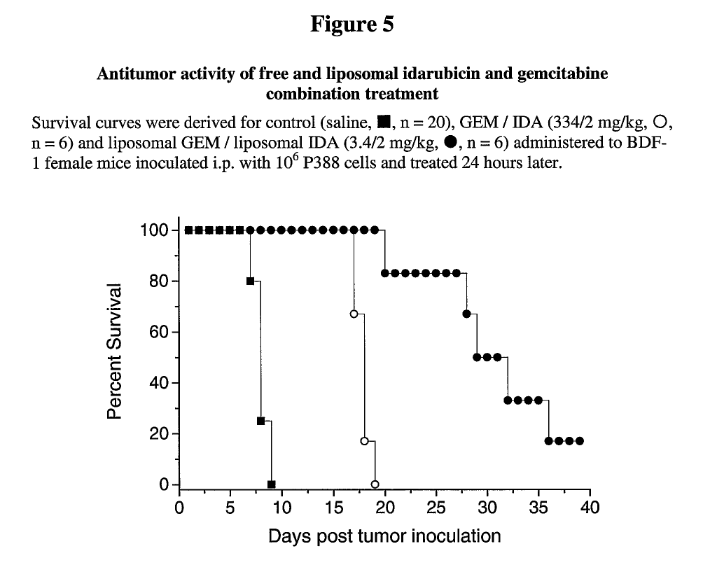

The survival of mice administered combinations of idarubicin/gemcitabine (IDA/GEM) and liposomal idarubicin/liposomal gemcitabine (LIDA/LGEM) are illustrated by the data shown in FIG. 5.

Table 3 also shows the effect of a study wherein mice were infected with varying numbers of P388 cells and median survival time was recorded. The results indicated that mice injected with 106, 105, 104, 103, 102 and 10 cells had median survival times of 8, 10.5, 11, 12, 15 and 17.5 days. By correlating median survival times from mice administered treatments, the log cell kill may be calculated. This analysis was not of substantial value of those groups exhibiting a log cell kill χ6, but when this was observed it correlated with groups having 1 or more long term survivors.

### SUMMARY OF RESULTS

The pharmacokinetic analysis comparing liposomal (DSPC/CH/DSPE-PEG2000; 50:45:5 mole ratio) and free gemcitabine indicated that significant increases in the mean plasma area-under-the curve (AUC), and plasma half-life (T1/2), area-under-the-moment curve (AUMC) and mean residence time (MRT), while total plasma clearance (Cl) was reduced with a mean plasma AUC and plasma half-life increase of 154-fold and 6.8-fold, respectively. Antitumor activity of liposomal gemcitabine in P388 murine model demonstrated improvements in median survival time at a 100-fold lower dose (compared to free drug).

Dose range finding studies were performed in non-tumor bearing mice to identify maximum tolerable dose, then 66% of MTD was chosen as the dose and combined with dose titrations. At the highest doses, the ratio was 2 mg/kg (3.8 μmole/kg) idarubicin and 334 mg/kg (1115 μmole/kg) gemcitabine or 2 mg/kg (3.8 μmole/kg) liposomal idarubicin and 3.4 mg/kg (6.4 μmole/kg) liposomal gemcitabine. Thus the fixed dose ratio of GEM/IDA was 167:1 wt/wt ratio and 298:1 mol/mol ratio. In turn, the fixed dose ratio of LGEM/LIDA was 1.7:1 wt/wt ratio and 1.7:1 mol/mol ratio.

1-32. (canceled)

33. A method to obtain a fixed ratio of two or more drugs in combination for therapeutic efficacy in vivo, comprising:
providing the maximum tolerated dose (MTD) of each of at least a first drug formulation and a second drug formulation as determined when said first and second drug formulations are administered separately, and fixing said ratio as the same set percentage of the MTD for each drug, to obtain said fixed ratio.

34. The method of claim 33 which further includes:
administering the fixed ratio to an animal model of a disease state; and
evaluating the effectiveness of said fixed ratio in treating said disease state in said animal model.

35. The method of claim 34, wherein the step of evaluating the effectiveness of the fixed ratio comprises determining whether said at least first and second drugs act non-antagonistically or synergistically in said animal model at said fixed ratio.

36. The method of claim 34, further comprising optimizing the dosage of each of said at least first and second drugs by selecting a dosage less than the MTD for each of said drugs while maintaining said fixed ratio.

37. The method of claim 33, wherein each of said drugs is in free form, or wherein each of said drugs is in liposomal form.

38. The method of claim 34, wherein said animal model is a murine cancer model.

39. The method of claim 33, wherein said MTD for each of said drugs has been defined using parameters comprising one or more toxicity end points defined by hematology, clinical chemistry, urinalysis, gross pathology or microscopic pathology.

40. The method of claim 33, wherein said MTD for each of said drugs has been defined by toxicity studies in said animal model based on the route of administration intended for humans.

41. A method to administer at least a first and second drug which method comprises administering said drugs in the fixed ratio as obtained by the method of claim 33.

42. The method of claim 41, wherein the first and second drugs are delivered in the same composition.

43. The method of claim 41, wherein at least one drug is associated with a particulate delivery vehicle.

44. A composition for delivering at least a first and second drug which comprises each of said first and second drug present in a fixed ratio as obtained by the method of claim 33.

45. The composition of claim 44, wherein at least one drug is associated with a particulate delivery vehicle.

46. The method of claim 45, wherein the delivery vehicle is a liposome.

47. The composition of claim 45, wherein both drugs are associated with particulate delivery vehicle.

48. The composition of claim 47, wherein both drugs are associated with the same drug delivery vehicle.

49. A composition comprising gemcitabine and idarubicin, wherein substantially all of the gemcitabine in the composition is associated with liposomes.
